(19) United States
(12) Patent Application Publication
(10) Pub. No.: US 2023/0389965 A1
(43) Pub. Date: Dec. 7, 2023

(54) SCREW ELEMENT OPTIMIZED FOR 3D PRINTING

(71) Applicant: MIMEO MEDICAL GMBH, Filderstadt (DE)

(72) Inventor: Frank HEUER, Filderstadt (DE)

(21) Appl. No.: 18/250,161

(22) PCT Filed: Oct. 21, 2021
(86) PCT No.: PCT/EP2021/079255

§ 371 Date: Apr. 21, 2023

(30) Foreign Application Priority Data
Oct. 21, 2020 (DE) 10 2020 006 464.7

(51) Int. Cl. A61B 17/70; A61B 17/86

(52) U.S. Cl. CPC A61B 17/7035; A61B 17/8615; B33Y 80/00

(57) ABSTRACT

A screw element for the fixation of bone components and bone fragments is disclosed having a shaft with an external thread and a longitudinal central axis extending along the shaft and defining distal and proximal directions. The screw element includes a continuous cannulation, the cannulation includes at least two laterally extending openings communicating with the cannulation, and that the openings are configured as a polygon in a side view.

### STATE OF THE ART

Various osteosynthesis devices such as screw elements for the fixation of bones or bone fragments are known in the prior art. Such bone screws are classically manufactured with CNC milling and turning machines. For the special bone thread geometries and above all for the different diameters of the screw elements, special threaded plates must be provided if they are to be manufactured in series production. This results in longer delivery times and higher costs. 3D printing offers a possible alternative to this, as all geometries can be produced without special tools, and thus without waiting for special tools. Significantly greater flexibility in geometry design is possible.

Pedicle screws, for example, are used as screw elements in the treatment of the spine. They are characterized in that they have two different threaded areas. Distally, a bone thread with coarse toothing and proximally with finer toothing is provided. A coarse toothing provides best hold in cancellous bone and a finer toothing provides higher hold at the cortex. Such screw elements, with fine and coarse toothing, are considered to be the current state of the art, as they combine the best holding characteristics on the spine. There is a thread transition area between the distal and proximal threads. In this thread transition area, another thread tooth is provided coming from distal between the threads of the distal thread, which then extends into the proximal area. This results in finer toothing in the proximal area. If such screw elements are conventionally manufactured with CNC milling and turning machines, the result is a spiral beginning of the additional thread profile, which increases along the peripheral direction in the radial direction. When the pedicle screw is screwed in, this spiral thread beginning of the thread transition area pushes into the bone to make room for the subsequent additional thread. In some cases, especially in weaker bone, this space-occupying process of the additional thread may cause the pedicle to unintentionally burst and create a fracture. This is due to the lack of thread precutting. It would therefore be desirable to provide a cutting edge for the additional thread. This is very difficult to do with conventional CNC manufacturing methods. 3D printing offers a very good alternative for this.

If bone screws are to be manufactured using the 3D printing process, further challenges can be expected. With an eye on cost efficiency in manufacturing, the post-processing of the parts after 3D printing must be optimized. A key optimization step is the reduction of all support structures required for construction, as they are costly and often have to be removed manually. Once the orientation is determined, there should be no straight surfaces or overhangs in orientation to save on exactly these support structures. This has implications for openings and other features, such as lateral fenestration openings and also the tool attachment point. Here, features corresponding to the state of the art are missing as to how these must be provided geometrically in order to have to use as few support structures as possible.

### REPRESENTATION OF THE INVENTION

Conventional CNC production of the screw element (1) with a cutting edge at the thread transition area is currently not possible or only possible with the highest technological effort. Additive manufacturing is therefore the method of choice. Additive manufacturing of metallic alloys, or 3D printing, uses the laser or electron beam melting process. All metallic alloys that are known and accepted as orthopedic implant materials are suitable materials. These include, for example, titanium, cobalt-chromium and stainless steel alloys.

The long-term success of a 3d-printed implant is highly dependent on its post-treatment. Targeted heat treatment and surface treatment are extremely important. Relevant literature is available on this subject, outlining the interrelationships of the post-treatments. Preferably, the 3D-printed parts are first stress-relieved between 500° C. and a maximum of 850° C. and then subjected to a hot isostatic pressing (HIP) process. The parts are then corundum blasted to remove loose particles from the surface. As another part of the surface treatment, a smoothing of the microstructures is performed. Here, chemical etching, which can optionally be assisted by galvanic voltage and/or mechanical stimulation, can be used to achieve an appropriate reduction in surface roughness. The aim is to remove the incompletely welded particles, since tensile stresses and micro-notches that occur here due to the incompletely welded particles can weaken the fatigue strength. After this process, a shot peening process is suitable to create residual compressive stresses on the implant surface. This additionally increases the fatigue strength.

When manufacturing with a 3d printing process, some design parameters have to be considered. On the one hand, a minimum wall thickness of all structures of at least 0.1 mm or better of at least 0.3 mm must be maintained, and on the other hand, gaps or slots must have a gap thickness of at least 0.1 mm, or better of at least 0.3 mm, so that during additive manufacturing, the gap also remains open and does not close unintentionally.

For the screw element (1) according to the invention, space-allocating coordinate references are defined, such as the proximal direction (101), the distal direction (102), which extend along a central axis (103). Extending outward from the central axis (103) the radial spread (104) is defined (FIG. 1).

To reduce the number of support structures during 3D printing, it is advantageous if the orientation (105) of the screw element (1) corresponds approximately to the direction of the central axis (103) and runs from proximal (101) to distal (102). A different orientation would require the screw element (1) to be built up at an angle in the 3D printer and a large number of lateral support structures would have to be provided. This would make production less cost-efficient. Optimally, the head area (10) is manufactured first. In this way, only the head has to be lined with support structures, and the diameter of the head (10) provides sufficient lateral support, especially for longer screw elements. With a larger support diameter, longer components do not have to be additionally supported in distal direction during 3D printing.

To avoid having to provide the entire surface of the head area (10) with support structures, it is preferable if the tool attachment point (90) is open in the proximal direction (101) and opens into a concentric cone-like recess (94) and has a substantially right-angled cone angle. Thus, the support structures can be reduced to a support structure ring (95) along the diameter of the tool attachment point.

For the same reason, it is also important that the tool attachment point (90) is bounded in the distal direction (102) by a wall (93) and that said wall (93) is inclined radially inwards in increasing distal direction (102) and that the cone angle formed by the wall is less than 120°. Preferably, this wall (93) has a substantially right-angled cone angle. This means that support structures for the base of the tool attachment point can also be reduced in this case. Removing any support structures from this base surface (93) would be a great challenge, as this base surface (93) is very difficult to reach for reworking.

According to a first embodiment, a screw element (1) for the fixation of bone components and bone fragments is described, which comprises a shaft (11), a neck area (20) and a head (10) located in proximal direction (101) and a tip (60) located in distal direction (102). The head (10) is preferably a lens, an oblique head or a spherical head. However, a combination of different curves and surfaces is also possible. The main feature of the head is that the head (10) has a larger outer diameter than the neck area (20). Preferably, the bone anchor has a tool attachment point (90) which is suitable for applying a torque. For minimally invasive treatment, it is advantageous if the bone anchor has a cannulation opening (80) passing completely through it, through which a surgical guide wire can be passed.

Bone screws which can be screwed to a bone are preferably used as screw elements (1). However, hooks, clamps, nails and other types of bone anchors can also be used. In the example of a screw element (1) given here, a bone screw with a shaft (11) and a bone thread (12) positioned on the shaft is presented. The thread (12) may have a finer toothing (30) proximally, at least sectionally, which is more suitable for a harder cortical bone. A distally tapering thread (60) with a cutting edge (61) at the bone anchor tip (60) is advantageous, so that the screw element (1) can self-tap into the bone when screwed in.

It is preferable if the screw element (1) is characterized in that the external thread (12) can be divided into a proximal threaded area (30) adjacent to the neck area (20) and extending in distal direction (102), and a distal threaded area (50) adjacent thereto, and a distal tip area (60) adjacent thereto, and the distal threaded area (50) merges into the proximal threaded area (30) in a transition zone (40), and the proximal threaded area (30) forms at least one additional thread (31, 32) which forms at least one cutting edge (41) within the transition zone (40).

This cutting edge (41) ensures a pre-cutting effect and does not cause any space displacement in the bone. This is of particular clinical advantage in weaker bone.

It is further preferable if at least one of the cutting edges (41, 61) is planar and oriented mainly in radial direction (104). Alternatively, a concave or convex surface is also possible for generating the respective cutting edge.

Different thread tooth courses and arrangements are possible as bone threads. For example, a thread with one tooth at the distal region can merge into a double or triple thread in the proximal region. It is also possible to envisage a double thread in the distal region, which merges into a quadruple or sixfold thread in the proximal direction (101). To simplify all figures, the preferred embodiment has been illustrated with a double thread in the distal region (50) and a quadruple thread at the proximal region (30) (FIGS. 1-4).

In the case of weak bone, such as osteopenia or osteoporosis, it may be necessary to additionally augment the bone anchor. This can be done with bone cement. Bone cement is preferably a polymer consisting of at least two components mixed together and injected in a liquid or paste-like state. After a few minutes, the bone cement hardens in the bone to form a plastic and bonds with the sponge-like bone structure. A polymethyl methacrylate cement is usually used. Alternatively, other media for delivery through the bone anchor are possible. It is possible that alternative media, such as pharmaceutically active media, or media containing cells, nutrients, or media serving as hereditary information carriers, or vaccines are administered through the bone anchor.

Optionally, the cannulation (80) comprises at least one or more laterally extending openings (70) communicating with the cannulation. Preferably, the openings are arranged in peripheral direction ring-like formation (71 or 72). In the case of more than one peripheral direction ring-like opening formation (71 and 72), the openings have different opening cross-sectional areas (710, 720) per formation. In the case of bone anchors (1) screwed into a bone, the lateral openings communicate with the surrounding bone tissue from the hollow chamber (80). They are configured to allow fluid injected into the bone anchor (1) to be delivered through the lateral openings into the surrounding tissue. A different cross-sectional area of the opening formations (710, 720) has the advantage that, due to the local pressure difference within the fluid, a similar volume flow is generated through all openings (71, 72). This is achieved because the openings (72) that are closer to the proximal direction (101) have a smaller cross-sectional area (720) than the openings (710) of the formation (71) that are further distal.

It is particularly advantageous for 3D printing if these lateral openings (70, 71, 72) are provided as polygons (700). Conventionally, such lateral openings (70) are drilled out concentrically. In 3D printing, concentric openings would create small overhangs, resulting in so-called dross formations (i.e. miniature dripstone-like formations). This would require manual post-processing, which is costly in volume production. Polygons offer an alternative, and are thus the preferred embodiment. The sloping panel elements of a polygon form a roof-like structure. Inclinations with an angle of about 45° can easily be produced by printing without support structures or dross formations.

Therefore, it is advantageous for the preferred embodiment that the lateral openings (70) are provided as a polygon (700). It is advantageous if the polygon (700) has at least one panel element (701, 702) which is mainly formed along the central axis (103). Furthermore, it is optimal if the distance between the panel elements (701 and 702) extending parallel to the central axis (103) is smaller than the cannulation diameter D82 at the outlet (83). Furthermore, the polygon (700) should have at least two panel elements (e.g. 703, 704, 705, 706), which in a side view are each oriented at an angle of from 25° to 65°, preferably from 35° to 55°, in particular from 40° to 50° with respect to the central axis (103), so that they can be produced at all along the defined orientation (105) using a 3D printing process.

According to the preferred embodiment, at least two panel elements (703, 704 or 705, 706) are oriented symmetrically with respect to the central axis (103). Alternative embodiments are also possible in which the polygon (700) is provided, for example, as a rhombus, parallelogram or with significantly more panel elements.

With 3D printing it is also possible to provide different roughnesses on the surface of the screw element (1) (FIG. 2). For example, it is possible to provide a higher surface roughness on the surfaces of the thread flanks (12) that are directed proximally (121) than on the surfaces of the same thread flanks that are directed distally (122). A higher roughness on the thread flanks in proximal projection direction (121) have the advantage of generating a higher friction between bone and screw element in pull-out direction and providing the screw element with a significantly higher pull-out strength. Smoother thread flanks in the distal projection direction (122) have the advantage that the screw element (1) can then still be easily screwed into the bone.

An alternative Screw element (1) for the fixation of bone components and bone fragments according to the invention comprises a shaft (11) with an external thread (12) and a longitudinal central axis (103) extending along the shaft (11), defining a distal (102) and a proximal (101) direction. The screw element (1) has a continuous cannulation (80). The cannulation (80) has at least two laterally extending openings (70) communicating with the cannulation (80, 83). The openings (70) are configured as a polygon (700) in a side view. The external thread (12) can be divided into a proximal threaded area (30) adjacent to the neck area (20) and extending in distal direction (102), and a distal threaded area (50) adjacent thereto, and a distal tip area (60) adjacent thereto. The distal threaded area (50) merges into the proximal threaded area (30) in a transition zone (40). The proximal threaded area (30) forms at least one additional thread (31, 32), which forms at least one cutting edge (41) within the transition zone (40). Another alternative screw element (1) according to the invention for the fixation of bone components and bone fragments comprises a shaft (11) with an external thread (12) and a longitudinal central axis (103) extending along the shaft (11), thereby defining a distal (102) and a proximal (101) direction. The external thread (12) can be divided into a proximal threaded area (30) adjacent to the neck area (20) and extending in distal direction (102), and a distal threaded area (50) adjacent thereto, and a distal tip area (60) adjacent thereto. The distal threaded area (50) merges into the proximal threaded area (30) in a transition zone (40). The proximal threaded area (30) forms at least one additional thread (31, 32), which forms at least one cutting edge (41) within the transition zone (40).

### BRIEF DESCRIPTION OF THE DRAWINGS SHOW

FIG. 1 an oblique view of the screw element according to the invention,

FIG. 2 a side view of the screw element according to the invention and a detailed view of the polygon-shaped lateral openings.

FIG. 3 side view and corresponding sectional view through the bone anchor according to the invention,

FIG. 4 two screw elements according to the invention in combination with u-shaped fork heads mounted with a connecting rod.

### DESCRIPTION OF THE PREFERRED EMBODIMENTS

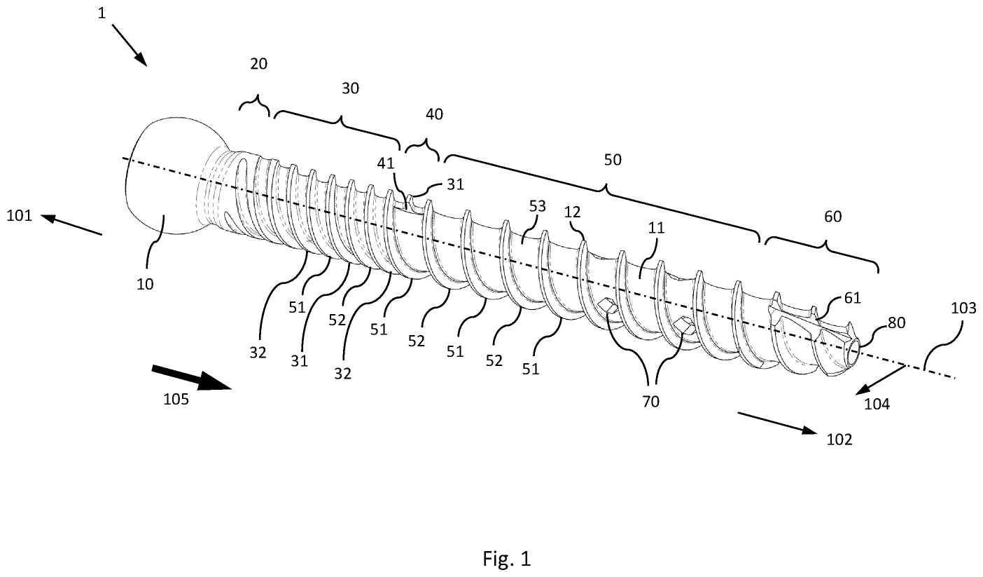

FIG. 1 shows the screw element (1) comprising a head area (10), a neck area (20) and a shaft area (11) with bone thread (12). Furthermore, it can be seen that the external thread (12) can be divided into a proximal threaded area (30) adjacent to the neck area (20) and extending in distal direction (102), and a distal threaded area (50) adjacent thereto, and a distal tip area (60) adjacent thereto, and the distal threaded area (50) merges into the proximal threaded area (30) in a transition zone (40), and the proximal threaded area (30) forms at least one additional thread (31, 32) which forms at least one cutting edge (41) within the transition zone (40).

Another cutting edge (61) is formed at the distal tip area (60). Optimally, the respective cutting edge (41, 61) is mainly planar in radial direction. Other surface geometries with convex or concave areas are also possible. Alternatively, recurring patterns with a pre-cutting effect, such as fluting or teeth, are also possible in the peripheral direction.

FIG. 1 shows a preferred embodiment of a screw element (1) which forms two separate thread teeth (51 and 52) in the distal area (50). This is a so-called double thread, whereby a larger pitch is achieved with the same number of thread teeth compared to a single thread. This reduces the number of turns required to implant such a screw element (1). The thread core or thread valleys (53) is located between the thread turns. In the proximal area (30), an additional thread tooth (31, 32) is provided between each of the distal threads (51, 52). The proximal threads (31, 32) have the same pitch as the distal threads (51 and 52). Two cutting edges (41, 42) are formed in the transition area (40), whereby the second cutting edge (42) cannot be shown due to the view. It is the beginning (not visible here) for the second proximal thread (32). The cutting edges (41, 42) have the great clinical advantage that fractures during implantation can be prevented in the future with such screw elements (1). If, alternatively, a different thread is provided, the number of cutting edges is increased or reduced accordingly.

FIG. 1 also shows that the orientation (105) of the screw element (1) corresponds substantially to the direction of the central axis (103) and runs from proximal (101) to distal (102). The advantages have already been described at the beginning.

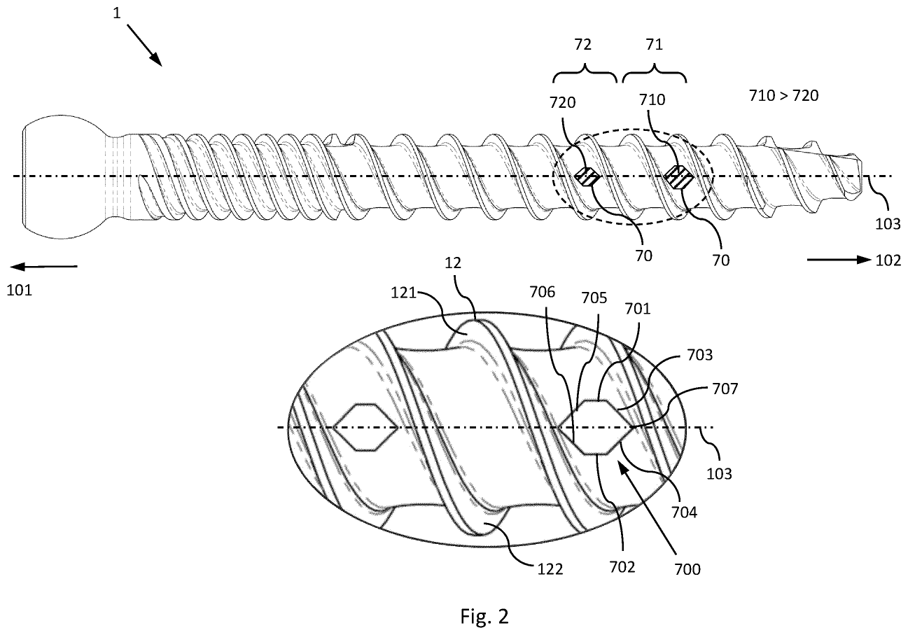

FIG. 2 shows a preferred embodiment of a screw element (1) in which the lateral fenestration openings (70) are formed as a polygon (700). The polygon (700) has at least one panel element (701, 702), which is mainly formed along the central axis (103). The distance between the panel elements (701 and 702) running parallel to the central axis (103) is smaller than the cannulation diameter D82 at the outlet (83). Furthermore, the polygon (700) has at least two panel elements (e.g. 703, 704, 705, 706) which, in a side view, are each oriented at an angle of from 25° to 65°, preferably from 35° to 55°, in particular from 40° to 50° with respect to the central axis (103). It is preferable if at least two of the panel elements (703, 704 or 705, 706) are oriented symmetrically with respect to the central axis (103). It is also possible for the panel elements (701-706) to merge into one another with the aid of curves (707).

FIG. 2 also shows that the lateral openings (70) are positioned in peripheral direction in ring-like formation (71 and/or 72) and, in the case of more than one peripheral direction ring-like formation (71 and 72), the openings (70) per formation have different opening cross-sectional areas (710, 720). Optimally, the opening cross-sectional area (710) of the distal formation (71) is larger than the opening cross-sectional area (720) of the proximal formation (72).

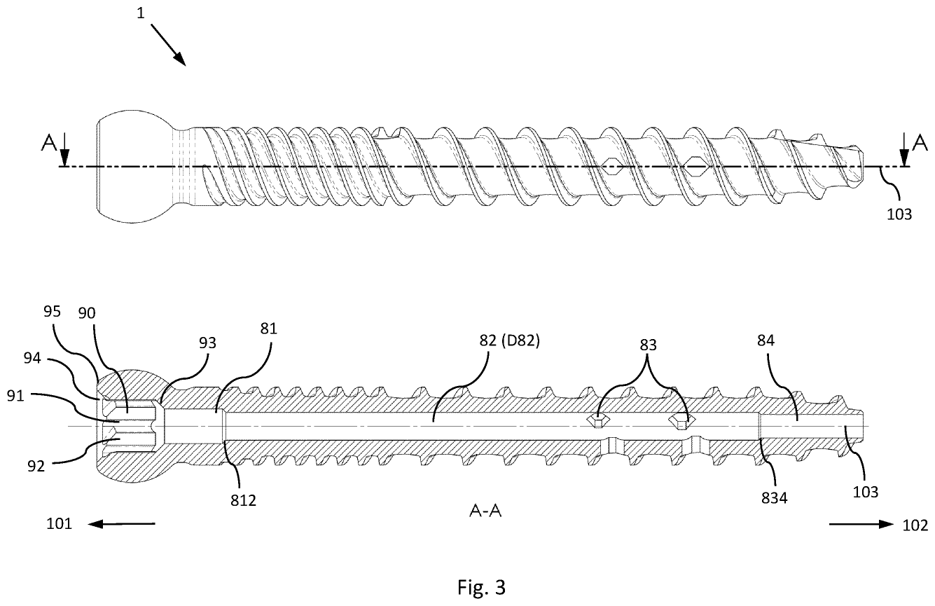

FIG. 3 shows a sectional view of the screw element (1). The interior of the head area (10) and the continuous cannulation (80) can be seen. The main feature of the head is that the head (10) has a larger outer diameter than the neck area (20). Preferably, the bone anchor has a tool attachment point (90) which is suitable for applying a torque. The torque for screwing in the bone anchor can thus be applied directly via the tool attachment point. This tool attachment point can have any profile (91, 92), such as a multi-tooth round, hexagon socket, cross recess, a simple slot or other toothing. According to the preferred embodiment, the tool attachment point (90) is positioned at the proximal end (101) and is bounded by a wall (93) in the distal direction (102). This wall (93) is formed as an inclination which extends radially inwards in increasing distal direction (102) and the cone angle formed by the wall is less than 120°. Optimally, the cone angle is substantially a right angle.

Furthermore, it can be seen that the tool attachment point (90) is open in proximal direction (101) and opens into a concentric cone-like recess (94) and has a substantially right-angled cone angle. The outer proximal ring functions as the support structure ring (95) described above.

FIG. 3 also shows the course of the cannulation opening (80). It is preferable if a section (81) with a slightly larger diameter is provided proximally, in which an application cannula can be inserted. Adjacent to this is the central section of the cannulation (82) with a diameter D82. The lateral openings (70) open into the cannulation (82) through corresponding outlets (83). It is advantageous if the cannulation diameter is reduced distally (102) in a distal cannulation section (84). The transitions (812, 834) between the different cannulation diameters ideally have a cone angle of less than 120°, preferably they are substantially right angled.

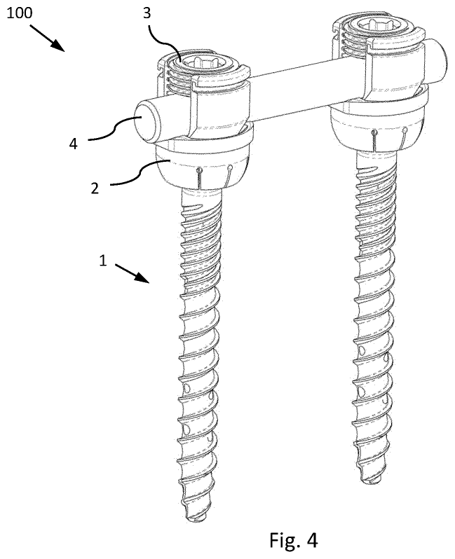

FIG. 4 shows two screw elements (1) according to the invention in combination with u-shaped fork heads (2) mounted with a connecting rod (4). The screw elements (1) have a proximal head area (10), which at least sectionally has a spherical segment, which is configured for providing a polyaxial pivotable connection with a fork head (2) that is u-shaped in a side view. After the connecting rod (4) has been inserted and the adjusting means (3) has been fixed, the screw elements (1) are connected to each other with angular stability. They form a rigid fixation, as used for example in spinal interventions.

1. A screw element for the fixation of bone components and bone fragments comprising a shaft with an external thread and a longitudinal central axis extending along the shaft and thereby defining a distal and a proximal direction, and the screw element comprises a continuous cannulation, the cannulation comprises at least two laterally extending openings communicating with the cannulation, wherein the openings are configured as a polygon in a side view.

2. The screw element according to the claim 1, wherein the polygon has at least one panel element formed mainly along the central axis.

3. The screw element according to claim 1, wherein the distance between the panel elements extending parallel to the central axis is smaller than the cannulation diameter D82 at the outlet.

4. The screw element according to claim 1, wherein the polygon has at least two panel elements which, in a side view, are each oriented at an angle of from 25° to 65°, with respect to the central axis.

5. The screw element according to claim 1, wherein at least two panel elements are symmetrically oriented with respect to the central axis.

6. The screw element according to claim 1, wherein the panel elements merge into one another with the aid of curves.

7. The screw element according to claim 1, wherein the lateral openings in peripheral direction are positioned in ring-like formation and in case of more than one in peripheral direction ring-like formation, the openings have different opening cross-sectional areas per formation.

8. The screw element according to claim 1, wherein the opening cross-sectional area of the distal formation is larger than the opening cross-sectional area of the proximal formation.

9. The screw element according to claim 1, wherein the lateral openings are positioned in the thread base.

10. The screw element according to claim 1, wherein the screw element additionally comprises a head, a neck area and a shaft area with bone thread and a tool attachment point is provided in the head.

11. The screw element according to claim 1, wherein the tool attachment point is bounded in the distal direction by a wall and said wall extends radially inward as an inclination in increasing distal direction and the cone angle formed by the wall is less than 120°.

12. The screw element according to claim 11, wherein the wall has a substantially right-angled cone angle.

13. The screw element according to claim 1, wherein the tool attachment point is open in proximal direction and opens into a concentric conical recess and has a substantially right-angled cone angle.

14. The screw element according to claim 1, wherein the external thread can be divided into a proximal threaded area adjacent to the neck area and extending in distal direction, and a distal threaded area adjacent thereto, and distal tip area adjacent thereto, and the distal threaded area merges into the proximal threaded area in a transition zone, and the proximal threaded area forms at least one additional thread which forms at least one cutting edge within the transition zone.

15. The screw element according to claim 1, wherein the distal tip area forms at least one cutting edge.

16. The screw element according to claim 14, wherein at least one of the cutting edges is planar and oriented substantially in radial direction.

17. The screw element according to claim 14, wherein at least one of the cutting edges has a concave surface which is oriented mainly in radial direction.

18. The screw element according to claim 14, wherein at least one of the cutting edges has a convex surface which is oriented mainly in radial direction.

19. The screw element according to claim 1, wherein the cannulation has, in proximal direction along the central axis, an at least sectionally cylindrical diameter extension configured to receive a cannula at least sectionally.

20. The screw element according to claim 1, wherein the head area comprises, at least sectionally, a spherical segment configured to provide a polyaxial pivotable connection with a fork head that is u-shaped in a side view.

21. The screw element according to claim 1, wherein the orientation of the screw element corresponds substantially to the direction of the central axis and extends from proximal to distal.

22. The screw element according to claim 1, wherein the external thread of the screw element has at least one surface facing mainly distally and at least one surface facing mainly proximally, and the proximally facing surface has a greater roughness than the distally facing surface.

23. The screw element for the fixation of bone components and bone fragments comprising a shaft with an external thread and a longitudinal central axis extending along the shaft and thereby defining a distal and a proximal direction, and the screw element comprises a continuous cannulation, the cannulation comprises at least two laterally extending openings communicating with the cannulation, wherein the openings are configured as a polygon in a side view, and that the external thread can be divided into a proximal threaded area adjacent to the neck area and extending in distal direction, and a distal threaded area adjacent thereto, and a distal tip area adjacent thereto, and the distal threaded area merges into the proximal threaded area in a transition zone, and the proximal threaded area forms at least one additional thread which forms at least one cutting edge within the transition zone.

24. The screw element for the fixation of bone components and bone fragments comprising a shaft with an external thread and a longitudinal central axis extending along the shaft and thereby defining a distal and a proximal direction and dividing the external thread into a proximal threaded area adjacent to the neck area and extending in distal direction, and a distal threaded area adjacent thereto, and a distal tip area adjacent thereto, and a distal threaded area adjacent thereto, and a distal tip area adjacent thereto, and the distal threaded area merges into the proximal threaded area in a transition zone, wherein the proximal threaded area forms at least one additional thread which forms at least one cutting edge within the transition zone.
